(19) United States
(12) Patent Application Publication
(10) Pub. No.: US 2025/0331572 A1
(43) Pub. Date: Oct. 30, 2025

(54) Aerosol-Generating Device with Controlling System and Corresponding Controlling Method

(71) Applicant: JT International SA, Geneva (CH)

(72) Inventors: Pier Paolo Monticone, Le Grand-Saconnex (CH); Layth Sliman Bouchuiguir, Bellevue (CH)

(73) Assignee: JT International SA, Geneva (CH)

(21) Appl. No.: 18/865,134

(22) PCT Filed: May 24, 2023
(86) PCT No.: PCT/EP2023/063905

§ 371 Date: Nov. 12, 2024

(30) Foreign Application Priority Data
May 24, 2022 (EP) 22175087.0

(51) Int. Cl. A24F 40/53; A24F 40/20; A24F 40/51; A24F 40/85

(52) U.S. Cl. CPC A24F 40/53; A24F 40/51; A24F 40/85; A24F 40/20

(57) ABSTRACT

An aerosol-generating device configured to receive a consumable article includes at least one optical element delimiting at least partially a heating cavity, at least one sensing system including an electromagnetic radiation detecting system configured to measure an intensity of an electromagnetic radiation passing through and/or reflected by the optical element, and a controlling system configured to monitor an optical parameter representative of the level of contamination of the heating cavity, based at least on the measured intensity, and trigger an alarm system depending on the parameter. An aerosol-generating system including the aerosol-generating device and an aerosol-generating article is also provided.

### TECHNICAL FIELD OF THE INVENTION

The present invention relates to the field of tobacco, in particular to reconstituted tobacco as well as aerosol-generating articles. The present invention further relates to electronic smoking devices, especially to electrically heated aerosol-generating systems.

### BACKGROUND OF THE INVENTION

Electronic cigarettes using aerosol-generating consumable articles have gained popularity in the recent years. There are mainly two types: liquid vaporizers and heated tobacco inhaler devices. Heated tobacco inhaler devices are referred to as “heat-not-burn” systems (HNB). They provide a more authentic tobacco flavour compared to electronic cigarettes which deliver an inhalable aerosol from heating of a liquid charge comprising aerosol formers, flavorants, and often nicotine. The HNB system's working principle is to heat a tobacco material comprising an aerosol-forming substance (such as glycerine and/or propylene glycol) which vaporises during heating and creates a vapour that extracts nicotine and flavour components from the tobacco material. The tobacco substance is heated to between 200 and 400° C., which is below the normal burning temperatures of a conventional cigarette. The inhaler device is typically a hand-held heater, which is configured to receive rod-shaped consumable articles.

When an aerosol-generating substrate, such as a tobacco substrate, is heated by a heating element in a heating cavity of the aerosol-generating device, volatile compounds are released. Such volatile compounds and also aerosol become deposited on surfaces of the aerosol-generating device. These substances are particularly deposited on side walls or bottom part of the heating cavity. Furthermore, these contamination substances may be accumulated and/or possibly partially removed by the friction of inserted aerosol-generating articles. Also, particles of the aerosol-generating articles, such as particles from their wrapper or particles of the substrate may also be deposited on the walls of a heater cavity. Thus, particles, contamination layers and dust collect generally inside the heating cavity of the device after the repetitive use of aerosol-generating articles.

The residual contamination layers and dust particles present a problem to the use of aerosol-generating devices and this in several aspects. First, residue layers and dust accumulation on the walls of the heating cavity can diminish or block the required air flow of the device. The contamination level may also affect the optimal flavour sensation of the aerosol. Indeed, the contamination layers and particles may impart an unpleasant or bitter flavour to a user. Also, the heating element may be damaged depending on how and where the contamination layers or particles are deposited. Although cleaning tools may be used to clean the heating cavity, damage may be produced by either too much or too few cleaning operations. Also, there exist some ways to reduce partially contamination, such as using a pyrolysis method, in which a heating element is heated to a temperature sufficiently high to burn any residues or deposits, however this is not always effective.

Up to now, it is usual to propose a cleaning frequency, for example by cleaning tools, that rely just on statistical knowledge of the mean contamination levels of heating cavities in function of the frequency of use of aerosol-articles with the device.

There is thus a need to provide a way to measure directly the level of contamination of a heating cavity, so that, once a threshold of a contamination is reached, a warning signal may be provided to the aerosol-generating system and so the user.

### SUMMARY OF THE INVENTION

The inventors of the present invention have found solutions to the above-discussed problems by providing an aerosol generating device that has an optoelectronic system to trigger a cleaning warning of the oven, also defined as heating cavity, of an aerosol-generating device. More specifically, the device of the invention allows providing an optical solution to detect and identify information on the contamination of the heating cavity by detecting and analyzing optical properties of the contamination substances deposited on the surface of said cavity.

In a first aspect, the invention thus relates to an aerosol-generating device comprising a heater body and a longitudinal heating cavity provided with an opening at one insertion end and adapted to receive at least part of an aerosol-generating article inserted through said opening.

The aerosol-generating device comprises:
at least one optical element, arranged onto or into said heater body, delimiting at least partially said cavity,
at least one sensing system comprising an electromagnetic radiation detecting system, comprising at least one detector, configured to measure an intensity of an electromagnetic radiation passing through and/or reflected by the optical element, and
a controlling system configured to monitor an optical parameter representative of the level of contamination of the heating cavity, based at least on said measured intensity, and trigger an alarm system depending on said parameter.

The invented device allows to provide a system and a method to measure oven dirtiness levels without having to rely on statistical estimations. Using an optical detection system allows to provide information on different possible optical characteristics of deposited dirt or dirt layers. Indeed, deposited dirt may for example alter at least one of the transmission, scattering, polarization, reflection of electromagnetic waves.

In an advantageous embodiment the at least one optical element comprises a window that is, at least partially, transparent to electromagnetic radiation. Integrating a window in a wall of a heater may be realized easily, without enhancing considerably the cost of an aerosol generating device. Windows may be small-sized elements.

The heater comprises preferably a heater body and heating wires or electrodes that surround the outside surface of the heater body. In variants, the heater body itself may generate the heat by for example imbedded wires or electrodes. The heater body may also be heated without contact, for example by a distant heating source such as an infrared source. The heater body has preferably a tubular shape but it cross section, defined perpendicular to its longitudinal axis, must not be necessarily circular. A tubular heater body may for example have a hexagonal cross section, providing flat lateral sides to which it is easier to integrate optical elements.

In an advantageous example, the window is at least partially made of glass, epoxy resin or sapphire. Using hard materials such as glass or sapphire allows to withstand extremely high temperatures, i.e. higher than 400° C. Some epoxy resins may also resist high temperatures such as 250° C. Using a transparent epoxy has the advantage that it may be cast as a window in an aperture of the heating cavity.

In an embodiment, the at least one optical element is located at the insertion end side of the cavity. This location is the most appropriate for devices with the current state of the art.

In an embodiment, the aerosol-generating device comprises a plurality of sensing systems provided along a longitudinal direction of the cavity.

In embodiments, the at least one sensing system further comprises an electromagnetic radiation emitting system configured to irradiate the optical element.

Advantageously, at least one electromagnetic radiation emitter and at least one electromagnetic radiation detector are located on the same side of the at least one optical element according to a transversal direction (X, Y) of the cavity.

In variants of execution, at least one electromagnetic radiation emitter and at least one electromagnetic radiation detecting system are located on opposite sides of the at least one optical element according to a transversal direction (X, Y) of the cavity.

In embodiments, the aerosol-generating device further comprising a reference intensity detecting system, comprising at least one reference detector, configured to measure a reference intensity of the electromagnetic radiation emitted by the at least one electromagnetic radiation emitter.

In embodiments, the aerosol-generating device further comprising a reference intensity detecting system, comprising at least one reference detector, configured to measure a reference intensity of an electromagnetic radiation outside the longitudinal cavity and preferably in the vicinity of the optical element, and wherein the controlling system is configured to monitor a ratio between said reference intensity and the measured intensity of the electromagnetic radiation passing through and/or reflected by the at least one optical element and trigger an alarm system depending on said ratio.

In variants, the aerosol-generating device comprises a beam splitter configured to reflect part of the electromagnetic radiation emitted by the electromagnetic radiation emitting system towards the reference intensity detecting system.

In examples of realization, the electromagnetic radiation emitting system comprises a semiconductor emitter emitting in a wavelength comprised between 300 nm and 10 μm, preferably between 300 nm and 5 μm.

In embodiments, the at least one electromagnetic radiation detector is configured to measure an intensity of infrared light issued by a heating element located around or in the cavity and passing through and/or being reflected by the optical element.

The invention also relates to an aerosol-generating system comprising an aerosol-generating device as described and an aerosol-generating article inserted at least partially in the cavity of said aerosol-generating device, with at least a portion of the aerosol-generating article facing at least one optical element.

The invention is also achieved by a method for controlling an aerosol-generating device as described. The method comprises at least the steps of:
a) measuring an intensity of an electromagnetic radiation passing through and/or detected by the at least one optical element,
b) monitoring a parameter representative of the level of contamination of the heating cavity, based at least on said measured intensity, and
c) triggering an alarm system depending on said parameter.

In an embodiment the parameter monitored in step b is representative of the level of dirt deposited on a surface of a heater body surrounding said cavity.

In variants, the method further comprises a step of irradiating the at least one optical element with an electromagnetic radiation. The radiation is preferably emitted by an electromagnetic radiation emitting system.

The method may also further comprise determining a reference intensity of the emitted electromagnetic radiation, and wherein in steps b) and c) the parameter is a ratio between said reference intensity and the measured intensity of the electromagnetic radiation passing through and/or reflected by the at least one optical element.

### BRIEF DESCRIPTION OF THE DRAWINGS

FIG. 1 shows a schematic representation of a partial longitudinal cross section of an aerosol-generating device comprising an optical detection system based on reflection of light from a window in the heating cavity, for detecting the contamination in a heating cavity.

FIG. 2 shows a schematic representation of a partial longitudinal cross section of an aerosol-generating device comprising an optical detection system based on transmission of light through the heating cavity for detecting the contamination in the heating cavity.

FIG. 3 and FIG. 4 show a schematic representation of a partial longitudinal cross section of an aerosol-generating device comprising an optical detection system comprising a reference detector for detecting the contamination in the heating cavity.

FIG. 5 shows a schematic representation of a partial longitudinal cross section of an aerosol-generating device comprising at least two optical detection systems for detecting the contamination in the heating cavity.

FIG. 6 shows a schematic representation of a partial longitudinal cross section of an aerosol-generating system wherein, upon illumination by an electromagnetic radiation emitting system, the illuminated area of the surface of an aerosol-generating article emits, by reflection and/or scattering effects, electromagnetic light.

FIG. 7 shows an optical detection system arranged in a heating cavity of a heater, with the system comprising at least a reflecting surface and a detector.

FIG. 8 shows an optical detection system arranged in a heating cavity of a heater, with the system comprising at least a microlens array and a detector.

FIG. 9 shows an optical detection system arranged in a heating cavity of a heater, with the system comprising a detector and at least a diffusor comprising structures to scatter electromagnetic waves.

FIG. 10 shows an optical detection system arranged in a heating cavity of a heater, with the system comprising a detector comprising a coating to the side of the heating cavity.

FIG. 11 shows an optical detection system arranged in a heating cavity of a heater, with the system comprising a detector and a lens arranged between the detector and the cavity.

FIG. 12 shows an optical detection system arranged in the heating cavity of a heater, with the system comprising a light trap configured to enhance the absorption effect by contaminating layers or contaminating particles that accumulate in the light trap.

FIGS. 13 and 14 show optical detection systems that comprise at least one optical fiber.

FIG. 15 illustrates an embodiment of an optical contamination detection system that is arranged at the entry opening of a heating cavity. In the embodiment of FIG. 15 a reference detector is arranged in proximity of a detector that is arranged in the cavity.

FIGS. 16 and 17 show an optical contamination sensor comprising a lens arranged in the side of a body of an aerosol generating device. The lens directs incident ambient light onto an aerosol-generating stick and a detector is configured to detect scattered light from the stick. FIGS. 16 and 17 show also a reference sensor that is arranged in the incident light beam.

FIG. 18 shows a heater body in contact with a heater element to provide infrared radiation into the cavity of a device.

### DETAILED DESCRIPTION OF THE INVENTION

The present invention will be described with respect to particular embodiments and with reference to the appended drawings, but the invention is not limited thereto. The drawings described are only schematic and are non-limiting. In the drawings, the size of some of the elements may be exaggerated and not drawn on scale for illustrative purposes. The dimensions and the relative dimensions do not correspond to actual reductions to practice of the invention.

The invention will be described in the following examples in relation to aerosol-generating consumable articles comprising a tobacco-containing charge of aerosol-generating material but the scope of the invention shall not be construed as limited to tobacco-based consumable articles but shall encompass any aerosol-generating consumable articles, such as smoking articles, heat-not-burn articles, e-liquid cartridges and cartomizers, which comprises an aerosol-generating substrate capable to generate an inhalable aerosol upon heating. Aerosol-generating consumable articles according to the current invention may or may not have a symmetry axis and may have any form or shape, such as an elongated, cylindrical shape, or a spherical shape, or the form of a beam.

In a first aspect the invention is realized by aerosol-generating device 1. The invention is further realized by an aerosol-generating system 2 that comprises said aerosol-generating device 1 and an aerosol-generating article 100 that is inserted in said aerosol-generating device 1.

As used herein, the term “aerosol-generating material” refers to a material capable of releasing volatile compounds upon heating, which can form an aerosol. The aerosol generated from aerosol-generating material may be visible or invisible and may include vapours (for example, fine particles of substances, which are in a gaseous state, that are ordinarily liquid or solid at room temperature) as well as gases and liquid droplets of condensed vapours.

The term “wrapper” is defined broadly as any structure or layer that protects and contains the charge of aerosol-generating material, and which allows to handle that material. A wrapper has an inner surface that may be in contact with the aerosol-generating material and has an outer surface away from the aerosol-generating material. A wrapper may preferably comprise a cellulose based material such as paper but may also be made of a biodegradable polymer or may be made of glass or a ceramic. The wrapper may be a porous material and may have a smooth or rough outer surface 5 and may be a flexible material or a hard material. A wrapper may constitute an optical opaque or partially transparent optical layer. In the case of paper, wrapper is a highly scattering layer and is, due to the fact that it is typically very thin, i.e. less than 100 μm, partially transparent in the visible, in the infrared, in the range of Teraherz radiation and may be partially transparent in the UV. A wrapper may also comprise apertures.

The term “heater” is a heating system to heat a substrate and comprises a cavity 200 also defined as heating cavity or oven, for introducing at least a portion of an article 100.

In a first aspect, the invention relates to an aerosol-generating device 1 comprising a longitudinal heating cavity 200 provided with an opening 201 at one insertion end 210 and adapted to receive at least part of an aerosol-generating article 100 inserted through said opening 201.

In an advantageous embodiment the heating cavity is defined by a tubular-shaped heating body. An embodiment of such heating body is illustrated in FIG. 18 In embodiments, the heating body is made of a metal. In variants, the heating body is heated by electrodes or heating wires that are arranged to the outside of the heater body and that are in thermal contact with the heating body. In variants, heating elements may be integrated into the heating body. In embodiments, the heating body may be a tubular shaped arrangement of heating elements such as heating wires or heating blades. The heating elements may be embedded in a protective layer such as an epoxy layer.

The aerosol-generating device 1 comprises;
at least one optical element 20, 22, 23, 24, 25, 26, 27, 28 delimiting at least partially said cavity 200;
at least one sensing system 10 comprising an electromagnetic radiation detecting system 14 configured to measure an intensity of an electromagnetic radiation passing through and/or being reflected by the optical element 20, 22, 23, 24, 25, 26, 27, 28.

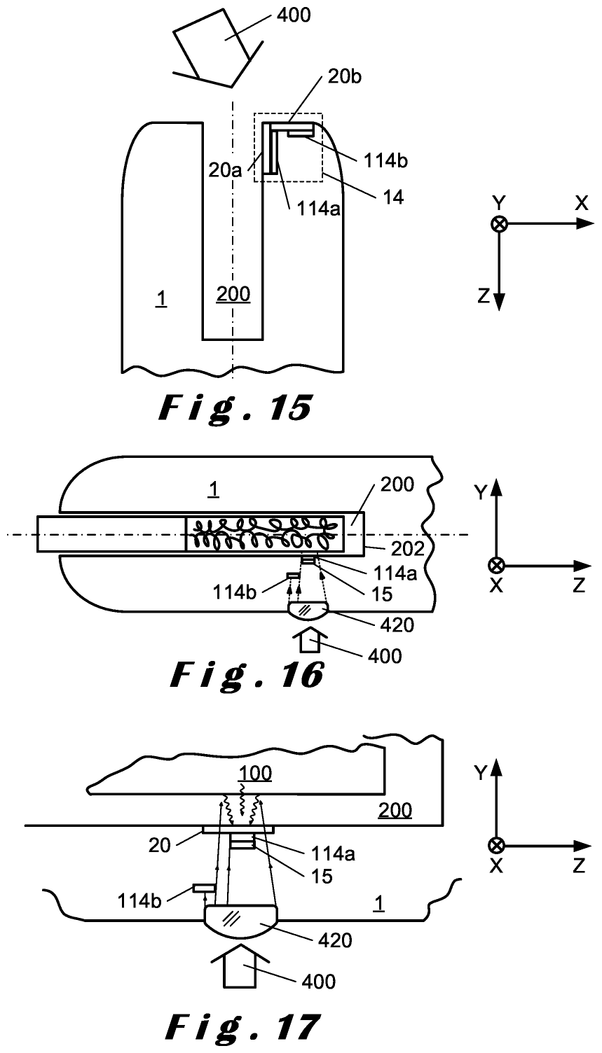

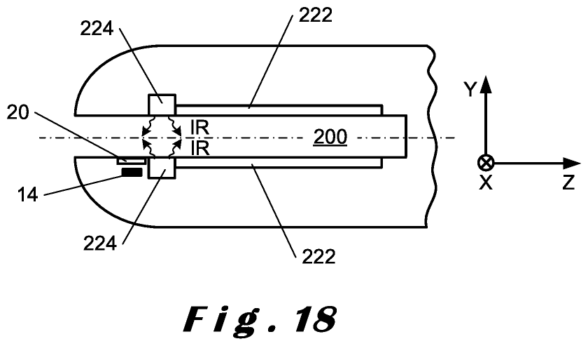

A device 1 has a front side 300 which is the side comprising the insertion opening 201 of the heating cavity 200. It is understood that said at least one sensing system 10 may be arranged to any position relative to said cavity 200, for example at or near to the insertion opening 201 of the device 1, as illustrated in FIG. 15 or it may be arranged to a lateral side of the device 1 and its cavity 200, as illustrated in FIGS. 16 and 17. In variants, a first optical detection system may be arranged in proximity of the opening 201 of the device 1. In variants, at least a second detection system may be arranged in the length or at the bottom side of the cavity 200, the bottom side being opposite to said opening 201.

An electromagnetic radiation detecting system 14 comprises at least one detector 114 that is also defined as a contamination detector. As explained further, the electromagnetic radiation detecting system 14 may comprise at least one reference detector 114b. An electromagnetic radiation detecting system 14 comprises an electronic circuit, for example to convert the electrical current provided by the detector 114 into an electrical voltage signal. The electromagnetic radiation detecting system 14 may comprise electrical processing means to treat that signal.

It is also understood that all embodiments disclosed herein may be combined together as far as it is technically feasible.

The aerosol-generating device 1 comprises also a controlling system 50 configured to monitor an optical parameter representative of the level of contamination of the heating cavity 200, based at least on said measured intensity, and trigger an alarm system depending on said parameter.

The invention allows to provide an aerosol-generating device 1, an aerosol-generating system and a method to measure oven dirtiness levels without having to rely on statistical estimations. Using an optical detection system allows to provide information on different possible optical characteristics of deposited dirt or dirt layers onto said heating body. Indeed, deposited dirt may for example alter at least one of the transmission, scattering, polarization, reflection of electromagnetic waves. In the case of a tubular-shaped heater body dirt or dirt layers may be deposited onto at least a portion of the inside surface of the heater body. Dirt or dirt layers may also be present in the heating cavity 200 and may be partially be in contact and/or adhere to the heater body. For example, thin residual tobacco parts or filaments may adhere to the heater body.

In an advantageous embodiment, illustrated in for example FIG. 1, the at least one optical element is a window 20 that is at least partially transparent to electromagnetic radiation, typically but not exclusive visible light or infrared radiation. The window 20 must not necessarily be a flat plate and may comprise at least one curved surface. Integrating a window 20 in a wall of a heater may be realized easily, without enhancing considerably the cost of an aerosol generating device. Windows may be small-sized elements, for example possibly having a maximal width of 1 mm and a thickness of possibly less than 0.5 mm. The window may close for example an aperture made into the wall of a heater. A window may comprise more than one window element that may be different window elements.

In an advantageous example, a window 20 is at least partially made of glass, epoxy resin or sapphire. Using hard materials such as glass or sapphire allows withstanding extremely high temperatures, i.e. higher than 400° C. Some epoxy resins may also resist high temperatures such as 250° C. Using a transparent epoxy has the advantage that it may be cast as a window 20 in an aperture of the heating cavity 200.

In an embodiment, the at least one optical element 20, 22, 23, 24, 25, 26, 27, 28 is located at the insertion end 210 side of the cavity 200. This location is the most appropriate for device 1 with the current state of practice.

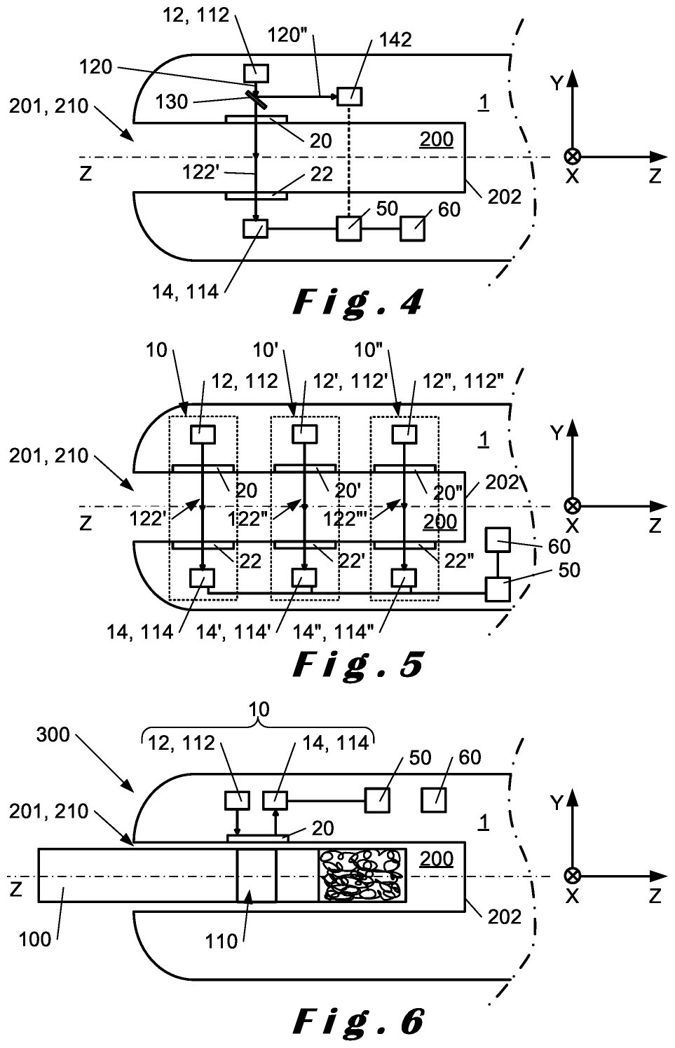

In an embodiment illustrated in FIG. 5, the aerosol-generating device 1 comprises a plurality of sensing systems 10, 10′, 10″ provided along a longitudinal direction Z of the cavity 200.

In embodiments, the at least one sensing system 10, 10′, 10″ further comprises an electromagnetic radiation emitting system 12, 12′, 12″, also called herein electromagnetic radiation source, configured to irradiate the optical element 20, 22, 23, 24, 25, 26, 27, 28. An electromagnetic radiation emitting system 12, 12′, 12″ may comprise a single emitter or, as illustrated in FIG. 2, at least two emitters 112, 112′. An electromagnetic radiation emitting system 12, 12′, 12″ comprises at least one electronic circuit to provide electrical power to the emitters 112, 112′, 112″.

In examples, an electromagnetic radiation emitting system 12, 12′, 12″ can comprise any source that provides electromagnetic radiation 120. Electromagnetic radiation 120 may be in the range of UV (ultraviolet), visible or infrared (IR) or terahertz radiation.

In examples of realization, the electromagnetic radiation emitting system 12, 12′, 12″ may comprise an emitter 112, 112′, 112″, for example a semiconductor emitter, emitting in a wavelength comprised between 300 nm and 10 μm, preferably between 300 nm and 5 μm. The electromagnetic radiation emitting system 12 may for example comprise a LED or a semiconductor laser emitting in visible light or in the infrared.

However, the electromagnetic radiation 120 must not necessarily be provided by a power-driven electromagnetic radiation source. The electromagnetic radiation emitting system 12 may be for example a surface portion of the heater or any hot section of the aerosol generating device 1 and/or or the consumable article 100, that provides a radiation of infrared light, as illustrated in FIG. 18.

In embodiments, as illustrated in FIG. 6, upon illumination by the electromagnetic radiation emitting system 12, the illuminated area of the surface of an aerosol-generating article 100 emits, by reflection and/or scattering effects, electromagnetic light. Such reemitted radiation may be provided by either the surface of an aerosol-generating article and/or by the contamination layer or dust particles accumulated on the inner surface of the cavity 200 of the heater. At least a portion 140 of the re-emitted radiation is directed to a detector 114 of a detecting system. An article 100 must not necessarily be introduced in the cavity 200 to perform the measurement of contamination levels. Indeed, the contamination substances influence intensity of the detected electromagnetic radiation 140 that is provided by the interaction of incident radiation 120 on the contamination layers or particles. Introducing an article 100 into the cavity 200 of a heater may increase the detected intensities as the surface of aerosol-generating articles are typically highly scattering surfaces that act as a partially reflecting surface. By using Terahertz frequencies, electromagnetic radiation may be transmitted through a whole diameter of an aerosol-generating articles. This means that a configuration, such as illustrated in FIGS. 2, 4 and 5, may be used even if an aerosol generating article 100 is present in the cavity 200 of the heater.

FIG. 6 illustrates an aerosol-generating system that comprises an aerosol-generating device 1 and an aerosol-generating article 100. The wrapper of the aerosol-generating article 100 is a highly light-scattering layer and allows to redirect emitted light from the emitter to the detector and so providing information on the contamination level of the window that separates the emitter and detector from the cavity. In variants, illustrated in FIG. 17, a detector 114 may be arranged to the cavity 200 without a window. In that case the contamination is produced directly onto the detector surface. An electromagnetic radiation detecting system 14 may comprise a single detector 114 or an array of detectors or may comprise a miniaturised vision system. The detecting system 14 may also comprise colour filters or a miniaturised spectrometer.

Advantageously, the electromagnetic radiation emitting system 12 and the electromagnetic radiation detecting system 14 are located on the same side of the at least one optical element according to a transversal direction (X) of the cavity. This is illustrated in the FIGS. 1, 3, 6, 7-12.

In variants of execution, illustrated in the FIGS. 2, 4, 5, the electromagnetic radiation emitting system 12 and the electromagnetic radiation detecting system 14 are located on opposite sides of the at least one optical element 20, 22, 23, 24, 25, 26, 27, 28 according to a transversal direction X of the cavity. Arrangements as illustrated in FIGS. 2, 4, 5 allow to provide a light beam 122, 122′, 122″ that traverses the cavity 200, allowing to provide a way to detect not only accumulated contamination layers but also dust or substrate particles in the space of the cavity 200.

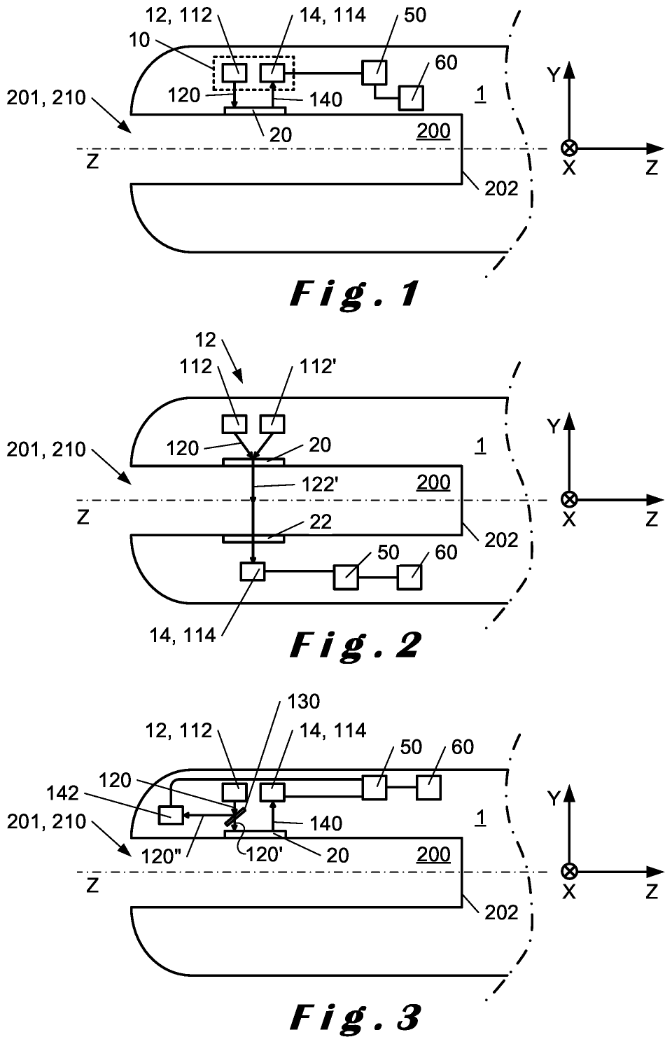

In advantageous embodiments, as illustrated in FIGS. 3 and 4, the aerosol-generating device 1 further comprises a reference intensity detecting system configured to measure a reference intensity of the electromagnetic radiation emitted by the electromagnetic radiation emitting system 12. In such a system the controlling system 50 is configured to monitor a ratio between said reference intensity and the measured intensity of the electromagnetic radiation passing through, or reflected by, the at least one optical element 20, 22, 23, 24, 25, 26, 27, 28 and trigger an alarm system depending on said ratio.

Providing a reference intensity allows to improve the reliability of the sensing system 10, as the detection may be rendered independent of for example aging effects of the detector, or intensity fluctuations of the emitting electromagnetic radiation source 12, 12′, 12″. For example, if infrared radiation is used as provided by the heater itself, the detection system may be calibrated and deliver a trigger signal that is independent of the intensity of the emitted radiation 120, and so the temperature of the heater.

In an embodiment, illustrated in FIG. 18, the heater body 222 of a heater may be in thermal contact with a heat retaining element 224 having another calorific coefficient, for example a ceramic ring 224. Such a ring may provide infrared light IR into the cavity. This infrared light IR may be redirected by the wrapper of an inserted article 100.

In another example of a system comprising an intensity reference, using for example a LED emitter 12, a signal may be provided that is related to the electrical driving of the LED, so that the detector system 14 detects the same intensity of light as was measured before contamination. Having to raise the intensity of the emitted light 120, and so the driving current of the LED, in order to achieve the same intensity as was detected before any contamination, allows to provide to an alarm system 60 an electrical signal that is directly related to the level of contamination of the heating cavity 200.

In examples of a sensing system 10 that comprises an intensity reference subsystem, illustrated in FIG. 3, an aerosol-generating device 1 comprises an electromagnetic wave beam splitter 130 configured to reflect part of the electromagnetic radiation 120 emitted by the electromagnetic radiation emitting system 112 towards a reference detector 142.

There are now further disclosed several variants that may be adapted to any of the embodiments as described herein.

For example, several means may be provided to enhance the level of intensity of re-emitted light 140 that may be detected. For example, a wrapper of an aerosol-generating article may comprise substances that enhance its reflectivity. For example, fluorescent materials may be integrated onto or into at least a portion of a wrapper and provide fluorescent or phosphorescent light effects. Also, in advantageous embodiments, illustrated in FIG. 6, an aerosol-generating article 100 may comprise at one of its surfaces at least one reflecting element 110 that enhances the intensity of reflected light from the surface an article.

A reflecting element 110 arranged to an article 100 may consist of or comprise at least one of:
reflecting elements, possibly a reflecting layer. A reflecting layer may be a coating or a reflector element fixed onto a wrapper, for example by gluing;
diffracting elements;
elements comprising meta-surfaces;
holographic elements;
polarization-sensitive elements.

A reflector 110 of electromagnetic radiation arranged on a surface of said wrapper may be realized by any physical or chemical means to a surface such as the wrapper of an aerosol-generating article 100. A reflecting element 110 may be arranged on a portion of a circumference of an article 100 or may be arranged on a complete circumference, as illustrated in FIG. 6. A reflector element 110 arranged onto an article 100 may be a polarization sensitive reflector.

A reflecting element 110 arranged to the aerosol-generating article of the invention may be configured to provide predetermined direct reflection effects such as providing, upon illumination by a light beam provided by a light source, a plurality of light beams that may have different spectra and/or different reflection angles. The reflected light beams may be diffracted light beams projected in any diffraction order. A reflector element 110 may provide a reflected light beam 140 that has a wide aperture so that a greater contamination area may be tested. This may be improved by using a lens such the one illustrated in the variant of FIG. 11.

It is generally understood that the optical sensing system 10 may comprise, but not exclusively:
refractive elements, for example single or compound lenses, prisms, beam splitters, Fresnel lenses;
reflective elements, for example flat or concave mirrors;
diffractive elements, for example diffractive lenses realized on a transparent substrate;
electrically addressable elements such as MEMS devices, for example MEMS shutter or MEMS micromirrors;
or a combination of such elements.

FIG. 7 shows a variant of optical detection system arranged in the cavity of a heater. In such variant the optical element is a reflecting surface or a mirror 23. The cumulated contamination of the reflecting surface will reduce its reflectivity and may be measured.

In another example, illustrated in FIG. 8, the sensing system 10 comprises at least a microlens array. Using a microlens array may allow to enhance the sensitivity of detection by the focusing effect of each element of the microlens array. Microlens arrays may also be arranged to an array of detectors, in a configuration wherein each microlens faces a detector of the array of detectors.

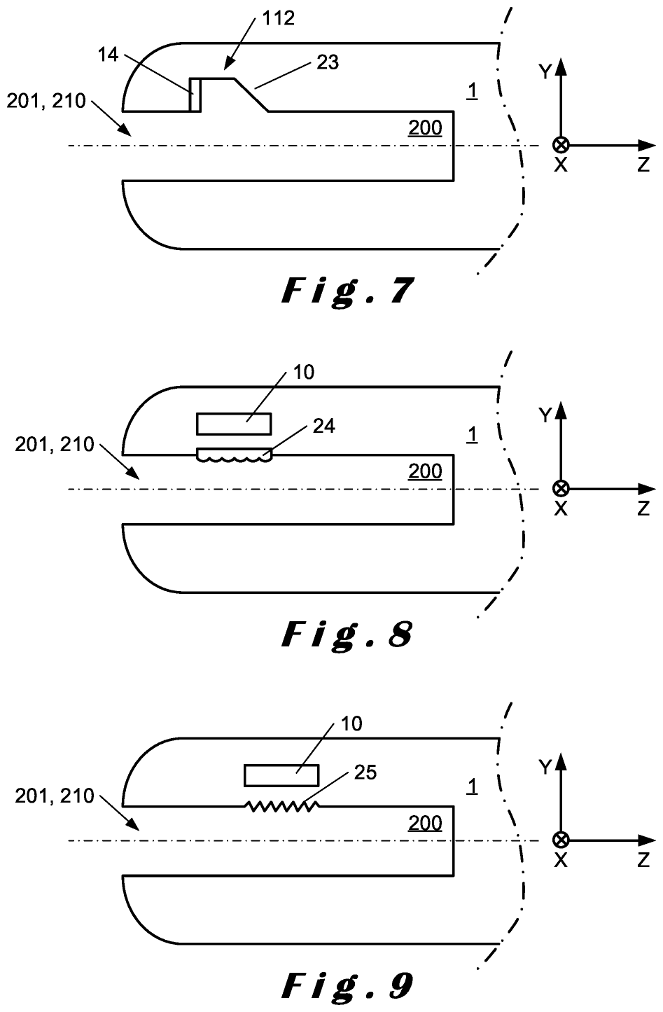

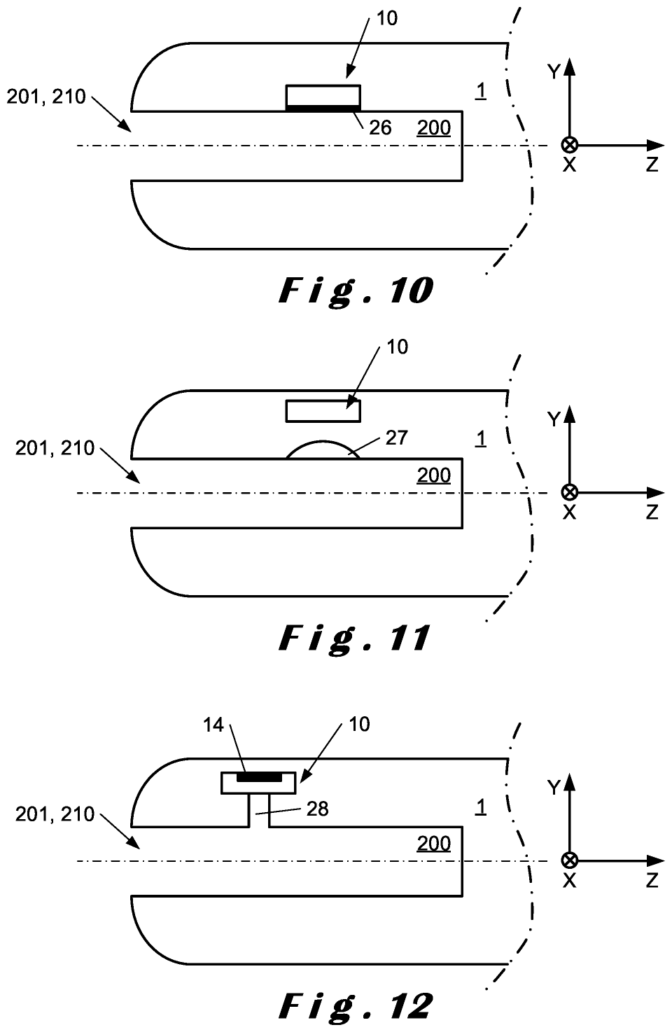

FIG. 9 shows another possible optical detection system arranged in the cavity 200 of a heater, wherein the system comprises a diffusor comprising structures to scatter electromagnetic waves. Using a diffuser allows to provide a smoothed-out intensity change effect that is provided by a larger contaminated surface

FIG. 10 shows an example of a simple optical detection system arranged in the cavity of a heater that relies on a detector comprising a coating 28 as the optical element. In variants the proception window of a detector may be used as the optical element from which contamination effects have to be determined.

FIG. 11 shows an optical detection system arranged in the cavity of a heater, and comprising a detector and a lens arranged between the detector and the cavity of the heater. Providing a lens 27 as the optical element allows to focus reemitted radiation onto a small detector that may be arranged away from the heater. Furthermore, using a lens 27 as an optical window allows to detect intensity variations due to contamination away from the wall of the heater, for example contamination by dust or substrate particles that stay suspended inside the heating cavity 200.

FIG. 12 shows an advantageous variant of a system comprising a light trap configured to enhance the absorption effect by contaminating layers or contaminating particles that accumulate in the light trap. An optical element in the form of a light trap 28, as illustrated in the example of FIG. 12, may amplify considerably the intensity variation effects due to contamination. In a sense, such a light trap 28 behaves like a waveguide due to the multiple reflections of radiation 140 that is directed to a detector 14. A detector in such a configuration may be arranged to the bottom of the cavity of the light trap, as illustrated in FIG. 12. In variants, a detector or multiple detectors may be arranged also to the side wall of such a light trap 28

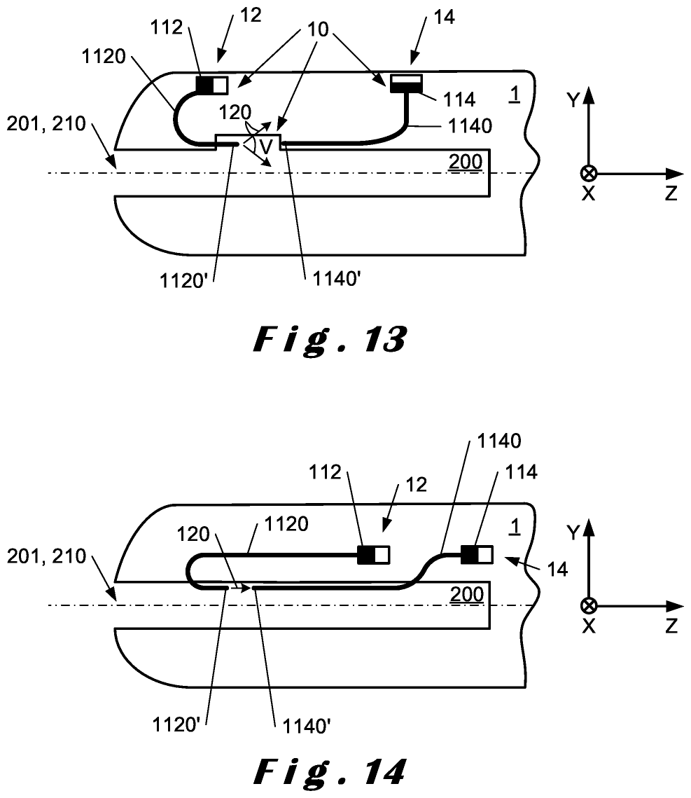

FIGS. 13 and 14 show examples of optical detection systems that comprise at least one optical fiber. Using waveguides, such as optical fibers or flat waveguides, allows for example to position an emitting system 12 and/or a detecting system 14 away from a hot surface. Furthermore, by using optical fibers that have a small emitting and/or collecting core allows to provide systems having a high contamination level detection sensitivity.

FIG. 13 shows an example of an emitting fiber 1120 that has an emitting edge 1120′ and a light collecting fiber 1140 that has a light collecting edge 1140′. Contamination of the heater cavity will reduce the transmitted intensity of the light beam 120 that traverses the space V between said emitting edge 1120′ and said light collecting edge 1140′. In an advantageous execution, illustrated in FIG. 14, the two fibers 1120 and 1140 may be arranged along and onto the wall of the heating cavity 200.

In other advantageous embodiments, the light source is not integrated into the device 1. Indeed, ambient light 400 such as direct or diffused sun light may be used to illuminate at least a portion of the surface of the cavity 200. This is illustrated in the embodiments of FIGS. 15, 16, 17.

FIG. 15 illustrates an embodiment of an optical contamination detection system that is arranged at a proximal end of a heating cavity 200. In the embodiment of FIG. 15, the detection system comprises an optical element in the form of a window 20a delimiting the cavity 200 at an insertion end side of the cavity, and a detecting system 14 comprising a contamination detector 114a configured to measure an intensity of an electromagnetic radiation passing through the window 20a and a reference detector 114b arranged in proximity of the contamination detector 114a and configured to measure an intensity of an electromagnetic radiation outside the body, in the vicinity of the insertion end. The window 20a and the reference detector 114b being very close to each other, an intensity of radiation measured by the contamination detector 114a and the reference detector 114b are comparable. By monitoring the intensity 11 detected by the contamination detector 114a and the intensity 12 detected by the reference detector 114b one may provide a signal I3=I1/I2 that is proportional to the absorption provided by contamination in proximity of the contamination detector 114a, for example a contamination layer deposited onto the contamination detector or to the window 20a that separates the contamination detector 114a from the cavity 200. In the case that the contamination detector 114a is arranged without window, it may have a protection layer to the side of the cavity 2w00. The protection layer may be for example a layer of SiO₂ or Al₂O₃.

FIGS. 16 and 17 show another configuration that does not require an integrated light source. In the aerosol-generating device 1 or aerosol-generating system, that includes an aerosol-generating article 100, the optical contamination sensor system comprises a lens 420 arranged at a side of a body of an aerosol generating device 1. The lens 420 directs incident ambient light 400 onto an aerosol-generating article 100 inserted in the longitudinal cavity 200 and a detector 114a, being a contamination detector, is configured to detect scattered light provided from the article 100. FIGS. 16 and 17 show also a reference sensor 114b that is arranged in the incident light beam. Preferably, as shown in FIG. 17, the reference detector 114b is inserted into the space defined by an incoming light beam 400. The contamination detector 114a may comprise to its back side a light shield 15, so that no light from the infalling lightbeam 400 may be detected by the back side, oriented to the infalling light side, of the detector 114a. In variants, not illustrated, there is no need for a window 20, and the surface of the contamination detector 114a may serve as the surface on which contamination is accumulated

In variants, ambient light may be provided by an external light source such as a pocked lamp or by a light emitter of a cellphone. The method of the invention may comprise a step of orienting said lamp or emitter in the direction of the contamination detector 114a.

It is understood that the invention is achieved as well by an aerosol-generating device 1 as an aerosol-generating system that comprises an aerosol-generating device 1 and an aerosol-generating article 100 inserted, at least partially, into the device 1.

The invention is also achieved by a method for controlling an aerosol- generating device 1 as described herein. The method comprises at least the steps of:
a) measuring an intensity of an electromagnetic radiation passing through and/or reflected by the at least one optical element 20, 22, 23, 24, 25, 26, 27, 28;
b) monitoring a parameter representative of the level of contamination of the heating cavity 200, based at least on said measured intensity, and
c) triggering an alarm system 60 depending on said parameter.

In variants the optical element 20 may be the front layer of a detector, such as a protection layer, for example an SiO₂ layer, of the front surface of a detector 114. The optical element must not be necessarily a separate optical component that is arranged in front of a detector 114.

In an embodiment, the method further comprises a step of irradiating the at least one optical element 20, 22, 23, 24, 25, 26, 27, 28 with an electromagnetic radiation emitting system 12.

In an embodiment, the method further comprises determining a reference intensity of the electromagnetic radiation emitting system 12, and in steps b) and c) the parameter is a ratio between said reference intensity and the measured intensity of the electromagnetic radiation passing through and/or reflected by the at least one optical element 20, 22, 23, 24, 25, 26, 27, 28.

In embodiments the method comprises a step of illuminating at least a portion of the surface of an aerosol-generating article 100 that is inserted into an aerosol-generating device 1, as illustrated in for example FIGS. 6, 16 and 17. In such an embodiment, the illumination step of the surface of the article 100 is followed by a detection step of at least a portion of the scattered light from the article 100.

In embodiments the illumination step may be provided by the emission of infrared light from the heater. In variants, an additional heating step may be provided. For example, it is possible to heat the heater shortly, for example during less than 1 second, so as to provide a temporal infrared flux to the detector. For example, the heater may be heated for less than 1 second above its normal operating temperature. Such an over-heating step may imply a temperature that is more than 50 degrees, possibly more than 100 degrees above the mean operation temperature. It is assumed that in such embodiment the heating flash is so short that the consumable product is still only heated and not burnt.

1. An aerosol-generating device comprising a heater body and a longitudinal heating cavity provided with an opening at one insertion end and adapted to receive at least part of an aerosol-generating article inserted through said opening,
wherein the aerosol-generating device comprises:
at least one optical element arranged onto or inside said heater body and delimiting at least partially said cavity,
at least one sensing system comprising at least one electromagnetic radiation detecting system, comprising at least one detector, configured to measure an intensity of an electromagnetic radiation passing through and/or being reflected by the at least one optical element, and
a controlling system configured to monitor an optical parameter representative of the level of contamination of the heating cavity, based at least on said measured intensity, and trigger an alarm system depending on said parameter.

2. The aerosol-generating device according to claim 1, wherein the at least one optical element comprises a window at least partially transparent to electromagnetic radiation.

3. The aerosol-generating device according to claim 1, wherein the at least one optical element and said at least one detector are located at the one insertion end of the cavity.

4. The aerosol-generating device according to claim 1, further comprising a plurality of sensing systems provided along a longitudinal direction of the cavity.

5. The aerosol-generating device according to claim 1, further comprising a reference intensity detecting configured to measure a reference intensity of an electromagnetic radiation outside the longitudinal heating cavity, and wherein the controlling system is configured to monitor a ratio between said reference intensity and the measured intensity of the electromagnetic radiation passing through and/or reflected by the at least one optical element and trigger an alarm system depending on said ratio.

6. The aerosol-generating device according to claim 1, wherein the at least one sensing system further comprises an electromagnetic radiation emitting system comprising at least one emitter configured to irradiate the at least one optical element.

7. The aerosol-generating device according to claim 6, wherein at least one of the at least one electromagnetic radiation emitter and at least one of the at least one detector of the at least one sensing system are located on the same side of the at least one optical element according to a transversal direction of the cavity.

8. The aerosol-generating device according to claim 6, wherein at least one of the at least one electromagnetic radiation emitter and at least one of the at least one detector (114), of the sensing system are located on opposite sides of the at least one optical element according to a transversal direction of the cavity.

9. The aerosol-generating device according to claim 5, wherein the at least one sensing system further comprises an electromagnetic radiation emitting system comprising at least one emitter configured to irradiate the at least one optical element, wherein the reference intensity detecting system is configured to measure a reference intensity of the electromagnetic radiation emitted by the at least one emitter.

10. The aerosol-generating device according to claim 6, wherein the at least one emitter comprises a semiconductor emitter emitting in a wavelength between 300 nm and 10 μm.

11. The aerosol-generating device according to claim 1, wherein the at least one detector is configured to measure an intensity of infrared light issued by a heating element located around or in the cavity and passing through and/or being reflected by the at least one optical element.

12. An aerosol-generating system comprising the aerosol-generating device according to claim 1 and an aerosol-generating article inserted at least partially in the cavity of said aerosol-generating device, with at least a portion of the aerosol-generating article facing at least one of the at least one optical element.

13. A method for controlling the aerosol-generating device according to claim 1, comprising at least the steps of:
a) measuring an intensity of an electromagnetic radiation passing through and/or reflected by the at least one optical element,
b) monitoring a parameter representative of the level of contamination of the heating cavity, based at least on said measured intensity, and
c) triggering an alarm system depending on said parameter.

14. The method according to claim 13, wherein the parameter monitored in step b) is representative of the level of dirt deposited on the surface of the heater body surrounding said cavity.

15. The method according to claim 13, further comprising a step of irradiating the at least one optical element with electromagnetic radiation.

16. The method according to claim 15, further comprising determining a reference intensity of the electromagnetic radiation, and wherein in steps b) and c) the parameter is a ratio between said reference intensity and the measured intensity of the electromagnetic radiation passing through and/or reflected by the at least one optical element.

17. The aerosol-generating device according to claim 6, wherein each of the at least one electromagnetic radiation emitter and each of the at least one detector of the at least one sensing system are located on the same side of the at least one optical element according to a transversal direction of the cavity.

18. The aerosol-generating device according to claim 6, wherein each of the at least one electromagnetic radiation emitter and each of the at least one detector of the sensing system are located on opposite sides of the at least one optical element according to a transversal direction of the cavity.

19. The aerosol-generating device according to claim 6, wherein the at least one emitter comprises a semiconductor emitter emitting in a wavelength between 300 nm and 5 μm.

20. The method according to claim 13, further comprising a step of irradiating the at least one optical element with visible light or infrared light.
